(19) United States
(12) Patent Application Publication
(10) Pub. No.: US 2011/0281894 A1
(43) Pub. Date: Nov. 17, 2011

(54) HEMIFUMARATE SALT

(75) Inventors: Per-Olov Bergström, Sodertalje (SE); Anna Minidis, Sodertalje (SE); Robert Ulf Johan Stranne, Sodertalje (SE); Mikael Wernersson, Sodertalje (SE)

(73) Assignee: ASTRAZENECA AB, Sodertalje (SE)

(21) Appl. No.: 13/105,569

(22) Filed: May 11, 2011

Related U.S. Application Data
(60) Provisional application No. 61/333,883, filed on May 12, 2010.

(51) Int. Cl. A61K 31/506; A61P 7/00; C07D 401/04; A61P 25/16; C07D 213/57; A61P 43/00; C07D 401/14; A61P 25/28

(52) U.S. Cl. 514/256; 544/333; 546/328; 546/330; 546/277.1

(57) ABSTRACT

The present invention relates to a hemifumarate salt of the compound (1S)-1-(2-(difluoromethyl)pyridin-4-yl)-4-fluoro-1-(3-(pyrimidin-5-yl)phenyl)-1H-isoindol-3-amine, Form A thereof and its pharmaceutical compositions. In addition, the present invention relates to therapeutic methods for the treatment and/or prevention of Aβ-related pathologies such as Downs syndrome, β-amyloid angiopathy such as but not limited to cerebral amyloid angiopathy or hereditary cerebral hemorrhage, disorders associated with cognitive impairment such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration.

### TECHNICAL FIELD OF THE INVENTION

The present invention relates to a hemifumarate salt of the compound (1S)-1-(2-(difluoromethyl)pyridin-4-yl)-4-fluoro-1-(3-(pyrimidin-5-yl)phenyl)-1H-isoindol-3-amine, Form A thereof and its pharmaceutical compositions. In addition, the present invention relates to therapeutic methods for the treatment and/or prevention of Aβ-related pathologies such as Downs syndrome, β-amyloid angiopathy such as but not limited to cerebral amyloid angiopathy or hereditary cerebral hemorrhage, disorders associated with cognitive impairment such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration.

### BACKGROUND

Several groups have identified and isolated aspartate proteinases that have β-secretase activity (Hussain et al., 1999; Lin et. al, 2000; Yan et. al, 1999; Sinha et. al., 1999 and Vassar et. al., 1999). β-secretase is also known in the literature as Asp2 (Yan et. al, 1999), Beta site APP Cleaving Enzyme (BACE) (Vassar et. al., 1999) or memapsin-2 (Lin et al., 2000). BACE was identified using a number of experimental approaches such as EST database analysis (Hussain et al. 1999); expression cloning (Vassar et al. 1999); identification of human homologs from public databases of predicted C. elegans proteins (Yan et al. 1999) and finally utilizing an inhibitor to purify the protein from human brain (Sinha et al. 1999). Thus, five groups employing three different experimental approaches led to the identification of the same enzyme, making a strong case that BACE is a β-secretase. Mention is also made of the patent literature: WO96/40885, EP871720, U.S. Pat. Nos. 5,942,400 and 5,744,346, EP855444, U.S. Pat. No. 6,319,689, WO99/64587, WO99/31236, EP1037977, WO00/17369, WO01/23533, WO0047618, WO00/58479, WO00/69262, WO01/00663, WO01/00665 and U.S. Pat. No. 6,313,268.

BACE was found to be a pepsin-like aspartic proteinase, the mature enzyme consisting of the N-terminal catalytic domain, a transmembrane domain, and a small cytoplasmic domain. BACE has an optimum activity at pH 4.0-5.0 (Vassar et al, 1999) and is inhibited weakly by standard pepsin inhibitors such as pepstatin. It has been shown that the catalytic domain minus the transmembrane and cytoplasmic domain has activity against substrate peptides (Lin et al, 2000). BACE is a membrane bound type 1 protein that is synthesized as a partially active proenzyme, and is abundantly expressed in brain tissue. It is thought to represent the major β-secretase activity, and is considered to be the rate-limiting step in the production of amyloid-β-protein (Aβ). It is thus of special interest in the pathology of Alzheimer's disease, and in the development of drugs as a treatment for Alzheimer's disease.

Aβ or amyloid-β-protein is the major constituent of the brain plaques which are characteristic of Alzheimer's disease (De Strooper et al, 1999). Aβ is a 39-42 residue peptide formed by the specific cleavage of a class 1 transmembrane protein called APP, or amyloid precursor protein. Cleavage of APP by BACE generates the extracellular soluble APPβ fragment and the membrane bound CTFβ (C99) fragment that is subsequently cleaved by γ-secretase to generate Aβ peptide.

Alzheimer's disease (AD) is estimated to afflict more than 20 million people worldwide and is believed to be the most common form of dementia Alzheimer's disease is a progressive dementia in which massive deposits of aggregated protein breakdown products—amyloid plaques and neurofibrillary tangles accumulate in the brain. The amyloid plaques are thought to be responsible for the mental decline seen in Alzheimer's patients.

The likelihood of developing Alzheimer's disease increases with age, and as the aging population of the developed world increases, this disease becomes a greater and greater problem. In addition to this, there is a familial link to Alzheimer's disease and consequently any individuals possessing the double mutation of APP known as the Swedish mutation (in which the mutated APP forms a considerably improved substrate for BACE) have a much higher risk of developing AD, and also of developing the disease at an early age (see also U.S. Pat. No. 6,245,964 and U.S. Pat. No. 5,877,399 pertaining to transgenic rodents comprising APP-Swedish). Consequently, there is also a strong need for developing a compound that can be used in a prophylactic fashion for these individuals.

The gene encoding APP is found on chromosome 21, which is also the chromosome found as an extra copy in Down's syndrome. Down's syndrome patients tend to develop Alzheimer's disease at an early age, with almost all those over 40 years of age showing Alzheimer's-type pathology (Oyama et al., 1994). This is thought to be due to the extra copy of the APP gene found in these patients, which leads to overexpression of APP and therefore to increased levels of Aβ causing the high prevalence of Alzheimer's disease seen in this population. Thus, inhibitors of BACE could be useful in reducing Alzheimer's-type pathology in Down's syndrome patients.

Drugs that reduce or block BACE activity should therefore reduce Aβ levels and levels of fragments of Aβ in the brain, or elsewhere where Aβ or fragments thereof deposit, and thus slow the formation of amyloid plaques and the progression of AD or other maladies involving deposition of Aβ or fragments thereof (Yankner, 1996; De Strooper and Konig, 1999). BACE is therefore an important candidate for the development of drugs as a treatment and/or prophylaxis of Aβ-related pathologies such as Downs syndrome, β-amyloid angiopathy such as but not limited to cerebral amyloid angiopathy or hereditary cerebral hemorrhage, disorders associated with cognitive impairment such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration.

It would therefore be useful to inhibit the deposition of Aβ and portions thereof by inhibiting BACE through inhibitors such as the compounds provided herein.

The therapeutic potential of inhibiting the deposition of Aβ has motivated many groups to isolate and characterize secretase enzymes and to identify their potential inhibitors (see, e.g., WO01/23533 A2, EP0855444, WO00/17369, WO00/58479, WO00/47618, WO00/77030, WO01/00665, WO01/00663, WO01/29563, WO02/25276, U.S. Pat. No. 5,942,400, U.S. Pat. No. 6,245,884, U.S. Pat. No. 6,221,667, U.S. Pat. No. 6,211,235, WO02/02505, WO02/02506, WO02/02512, WO02/02518, WO02/02520, WO02/14264, WO05/058311, WO05/097767, WO06/041404, WO06/041405, WO06/0065204, WO06/0065277, US2006287294, WO06/138265, US20050282826, US20050282825, US20060281729, WO06/138217, WO06/138230, WO06/138264, WO06/138265, WO06/138266, WO06/099379, WO06/076284, US20070004786, US20070004730, WO07/011,833, WO07/011,810, US20070099875, US20070099898, WO2007/149033, WO07/058,601, WO07/058,581, WO07/058,580, WO07/058,583, WO07/058,582, WO07/058,602, WO07/073,284, WO07/049,532, WO07/038,271, WO07/016,012, WO07/005,366, WO07/005,404 and WO06/0009653.

(1S)-1-(2-(Difluoromethyl)pyridin-4-yl)-4-fluoro-1-(3-(pyrimidin-5-yl)phenyl)-1H-isoindol-3-amine is disclosed in WO2010/056196.

### BRIEF DESCRIPTION OF THE DRAWINGS

FIG. 1 is an X-ray powder diffractogram of (1S)-1-(2-(difluoromethyl)pyridin-4-yl)-4-fluoro-1-(3-(pyrimidin-5-yl)phenyl)-1H-isoindol-3-amine hemifumarate Form A (° 2 theta).

FIG. 2 is an X-ray powder diffractogram of (1S)-1-(2-(difluoromethyl)pyridin-4-yl)-4-fluoro-1-(3-(pyrimidin-5-yl)phenyl)-1H-isoindol-3-amine hemifumarate Form A (d-spacing).

### OUTLINE OF THE INVENTION

The present invention relates to a hemifumarate salt of the compound (1S)-1-(2-(difluoromethyl)pyridin-4-yl)-4-fluoro-1-(3-(pyrimidin-5-yl)phenyl)-1H-isoindol-3-amine.

One embodiment of the present invention is (1S)-1-(2-(difluoromethyl)pyridin-4-yl)-4-fluoro-1-(3-(pyrimidin-5-yl)phenyl)-1H-isoindol-3-amine hemifumarate Form A.

One embodiment of the present invention is (1S)-1-(2-(difluoromethyl)pyridin-4-yl)-4-fluoro-1-(3-(pyrimidin-5-yl)phenyl)-1H-isoindol-3-amine hemifumarate Form A, characterized in providing an X-ray powder diffraction (XRPD) pattern, exhibiting substantially the following main peaks with d-values:

| d-spacing | Relative |
|---|---|
| [Å] | intensity |
| 4.37 | vs |

One embodiment of the present invention is (1S)-1-(2-(difluoromethyl)pyridin-4-yl)-4-fluoro-1-(3-(pyrimidin-5-yl)phenyl)-1H-isoindol-3-amine hemifumarate Form A, characterized in providing an X-ray powder diffraction pattern, exhibiting substantially the following main peaks with d-values:

| d-spacing | Relative |
|---|---|
| [Å] | intensity |
| 8.2 | s |
| 5.3 | s |
| 4.92 | s |
| 4.54 | s |
| 4.37 | vs |

One embodiment of the present invention is (1S)-1-(2-(difluoromethyl)pyridin-4-yl)-4-fluoro-1-(3-(pyrimidin-5-yl)phenyl)-1H-isoindol-3-amine hemifumarate Form A, characterized in providing an X-ray powder diffraction pattern, exhibiting substantially the following main peaks with d-values:

| d-spacing | Relative |
|---|---|
| [Å] | intensity |
| 8.7 | m |
| 8.2 | s |
| 7.3 | m |
| 6.7 | m |
| 6.4 | m |
| 6.2 | w |
| 5.9 | m |
| 5.3 | s |
| 4.92 | s |
| 4.54 | s |
| 4.37 | vs |
| 4.14 | m |

One embodiment of the present invention is (1S)-1-(2-(difluoromethyl)pyridin-4-yl)-4-fluoro-1-(3-(pyrimidin-5-yl)phenyl)-1H-isoindol-3-amine hemifumarate Form A, characterized in providing an X-ray powder diffraction pattern, exhibiting substantially the following main peaks with d-values:

| d-spacing | Relative |
|---|---|
| [Å] | intensity |
| 13.3 | vw |
| 8.7 | m |
| 8.2 | s |
| 7.3 | m |
| 6.7 | m |
| 6.4 | m |
| 6.2 | w |
| 5.9 | m |
| 5.6 | w |
| 5.3 | s |
| 4.92 | s |
| 4.54 | s |
| 4.37 | vs |
| 4.14 | m |

The relative intensities are derived from diffractograms measured with variable slits.

The measured relative intensities vs. the strongest peak are given as very strong (vs) above 50%, as strong (s) between 25 and 50%, as medium (m) between 10 and 25%, as weak (w) between 5 and 10% and as very weak (vw) under 5% relative peak height. It will be appreciated by a person skilled in the art that the XRPD intensities may vary between different samples and different sample preparations for a variety of reasons including preferred orientation. It will also be appreciated by a person skilled in the art that smaller shifts in the measured Angle and hence the d-spacing may occur for a variety of reasons including variation of sample surface level in the diffractometer.

The term (15)-1-(2-(difluoromethyl)pyridin-4-yl)-4-fluoro-1-(3-(pyrimidin-5-yl)phenyl)-1H-isoindol-3-amine hemifumarate also encompasses all solvates and co-crystals thereof.

In another aspect of the invention, there is provided a pharmaceutical composition comprising as active ingredient a therapeutically effective amount of (1S)-1-(2-(difluoromethyl)pyridin-4-yl)-4-fluoro-1-(3-(pyrimidin-5-yl)phenyl)-1H-isoindol-3-amine hemifumarate in association with pharmaceutically acceptable excipients, carriers or diluents.

In another aspect of the invention, there is provided (1S)-1-(2-(difluoromethyl)pyridin-4-yl)-4-fluoro-1-(3-(pyrimidin-5-yl)phenyl)-1H-isoindol-3-amine hemifumarate for use as a medicament.

In another aspect of the invention, there is provided use of (1S)-1-(2-(difluoromethyl)pyridin-4-yl)-4-fluoro-1-(3-(pyrimidin-5-yl)phenyl)-1H-isoindol-3-amine hemifumarate, as a medicament for treating or preventing an Aβ-related pathology.

In another aspect of the invention, there is provided use of (1S)-1-(2-(difluoromethyl)pyridin-4-yl)-4-fluoro-1-(3-(pyrimidin-5-yl)phenyl)-1H-isoindol-3-amine hemifumarate, as a medicament for treating or preventing an Aβ-related pathology, wherein said Aβ-related pathology is Downs syndrome, a β-amyloid angiopathy, cerebral amyloid angiopathy, hereditary cerebral hemorrhage, a disorder associated with cognitive impairment, MCI (“mild cognitive impairment”), Alzheimer Disease, memory loss, attention deficit symptoms associated with Alzheimer disease, neurodegeneration associated with Alzheimer Disease, dementia of mixed vascular origin, dementia of degenerative origin, pre-senile dementia, senile dementia, dementia associated with Parkinson's disease, progressive supranuclear palsy or cortical basal degeneration.

In another aspect of the invention, there is provided use of (1S)-1-(2-(difluoromethyl)pyridin-4-yl)-4-fluoro-1-(3-(pyrimidin-5-yl)phenyl)-1H-isoindol-3-amine hemifumarate, as a medicament for treating or preventing Alzheimer Disease.

In another aspect of the invention, there is provided use of (1S)-1-(2-(difluoromethyl)pyridin-4-yl)-4-fluoro-1-(3-(pyrimidin-5-yl)phenyl)-1H-isoindol-3-amine hemifumarate, in the manufacture of a medicament for treating or preventing an Aβ-related pathology.

In another aspect of the invention, there is provided use of (1S)-1-(2-(difluoromethyl)pyridin-4-yl)-4-fluoro-1-(3-(pyrimidin-5-yl)phenyl)-1H-isoindol-3-amine hemifumarate, in the manufacture of a medicament for treating or preventing an Aβ-related pathology, wherein said Aβ-related pathology is Downs syndrome, a β-amyloid angiopathy, cerebral amyloid angiopathy, hereditary cerebral hemorrhage, a disorder associated with cognitive impairment, MCI (“mild cognitive impairment”), Alzheimer Disease, memory loss, attention deficit symptoms associated with Alzheimer disease, neurodegeneration associated with Alzheimer disease, dementia of mixed vascular origin, dementia of degenerative origin, pre-senile dementia, senile dementia, dementia associated with Parkinson's disease, progressive supranuclear palsy or cortical basal degeneration.

In another aspect of the invention, there is provided use of (1S)-1-(2-(difluoromethyl)pyridin-4-yl)-4-fluoro-1-(3-(pyrimidin-5-yl)phenyl)-1H-isoindol-3-amine hemifumarate, in the manufacture of a medicament for treating or preventing Alzheimer's Disease.

In another aspect of the invention, there is provided a method of inhibiting activity of BACE comprising contacting said BACE with (1S)-1-(2-(difluoromethyl)pyridin-4-yl)-4-fluoro-1-(3-(pyrimidin-5-yl)phenyl)-1H-isoindol-3-amine hemifumarate.

In another aspect of the invention, there is provided a method of treating or preventing an Aβ-related pathology in a patient in need thereof, comprising administering to said patient a therapeutically effective amount of (1S)-1-(2-(difluoromethyl)pyridin-4-yl)-4-fluoro-1-(3-(pyrimidin-5-yl)phenyl)-1H-isoindol-3-amine hemifumarate.

In another aspect of the invention, there is provided a method of treating or preventing an Aβ-related pathology in a patient in need thereof, comprising administering to said patient a therapeutically effective amount of (1S)-1-(2-(difluoromethyl)pyridin-4-yl)-4-fluoro-1-(3-(pyrimidin-5-yl)phenyl)-1H-isoindol-3-amine hemifumarate, wherein said Aβ-related pathology is Downs syndrome, a β-amyloid angiopathy, cerebral amyloid angiopathy, hereditary cerebral hemorrhage, a disorder associated with cognitive impairment, MCI (“mild cognitive impairment”), Alzheimer Disease, memory loss, attention deficit symptoms associated with Alzheimer disease, neurodegeneration associated with Alzheimer disease, dementia of mixed vascular origin, dementia of degenerative origin, pre-senile dementia, senile dementia, dementia associated with Parkinson's disease, progressive supranuclear palsy or cortical basal degeneration.

In another aspect of the invention, there is provided a method of treating or preventing Alzheimer's Disease in a patient in need thereof, comprising administering to said patient a therapeutically effective amount of (1S)-1-(2-(difluoromethyl)pyridin-4-yl)-4-fluoro-1-(3-(pyrimidin-5-yl)phenyl)-1H-isoindol-3-amine hemifumarate.

In some embodiments, the present invention provides a method of inhibiting activity of BACE comprising contacting the BACE with (1S)-1-(2-(difluoromethyl)pyridin-4-yl)-4-fluoro-1-(3-(pyrimidin-5-yl)phenyl)-1H-isoindol-3-amine hemifumarate. BACE is thought to represent the major β-secretase activity, and is considered to be the rate-limiting step in the production of amyloid-β-protein (Aβ). Thus, inhibiting BACE through inhibitors such as the compounds provided herein would be useful to inhibit the deposition of Aβ and portions thereof. Because the deposition of Aβ and portions thereof is linked to diseases such Alzheimer Disease, BACE is an important candidate for the development of drugs as a treatment and/or prophylaxis of Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration.

In some embodiments, the present invention provides a method for the prophylaxis of Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration comprising administering to a mammal (including human) a therapeutically effective amount of (1S)-1-(2-(difluoromethyl)pyridin-4-yl)-4-fluoro-1-(3-(pyrimidin-5-yl)phenyl)-1H-isoindol-3-amine hemifumarate.

In some embodiments, the present invention provides a method of treating or preventing Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration by administering to a mammal (including human) (1S)-1-(2-(difluoromethyl)pyridin-4-yl)-4-fluoro-1-(3-(pyrimidin-5-yl)phenyl)-1H-isoindol-3-amine hemifumarate and a cognitive and/or memory enhancing agent.

In some embodiments, the present invention provides a method of treating or preventing Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration by administering to a mammal (including human) (1S)-1-(2-(difluoromethyl)pyridin-4-yl)-4-fluoro-1-(3-(pyrimidin-5-yl)phenyl)-1H-isoindol-3-amine hemifumarate and a choline esterase inhibitor or anti-inflammatory agent.

In some embodiments, the present invention provides a method of treating or preventing Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration, or any other disease, disorder, or condition described herein, by administering to a mammal (including human) a compound of the present invention and an atypical antipsychotic agent. Atypical antipsychotic agents includes, but not limited to, Olanzapine (marketed as Zyprexa), Aripiprazole (marketed as Abilify), Risperidone (marketed as Risperdal), Quetiapine (marketed as Seroquel), Clozapine (marketed as Clozaril), Ziprasidone (marketed as Geodon) and Olanzapine/Fluoxetine (marketed as Symbyax).

In some embodiments, the mammal or human being treated with (1S)-1-(2-(difluoromethyl)pyridin-4-yl)-4-fluoro-1-(3-(pyrimidin-5-yl)phenyl)-1H-isoindol-3-amine hemifumarate has been diagnosed with a particular disease or disorder, such as those described herein. In these cases, the mammal or human being treated is in need of such treatment. Diagnosis, however, need not be previously performed.

The definitions set forth in this application are intended to clarify terms used throughout this application. The term “herein” means the entire application.

As used herein, “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The anti-dementia treatment defined herein may be applied as a sole therapy or may involve, in addition to the compound of the invention, conventional chemotherapy. Such chemotherapy may include one or more of the following categories of agents: acetyl cholinesterase inhibitors, anti-inflammatory agents, cognitive and/or memory enhancing agents or atypical antipsychotic agents.

Such conjoint treatment may be achieved by way of the adjunct, concurrent, simultaneous, sequential or separate dosing of the individual components of the treatment. Such combination products employ the compounds of this invention.

Additional conventional chemotherapy may include one or more of the following categories of agents:

(i) antidepressants such as agomelatine, amitriptyline, amoxapine, bupropion, citalopram, clomipramine, desipramine, doxepin duloxetine, elzasonan, escitalopram, fluvoxamine, fluoxetine, gepirone, imipramine, ipsapirone, maprotiline, nortriptyline, nefazodone, paroxetine, phenelzine, protriptyline, ramelteon, reboxetine, robalzotan, sertraline, sibutramine, thionisoxetine, tranylcypromaine, trazodone, trimipramine, venlafaxine and equivalents and pharmaceutically active isomer(s) and metabolite(s) thereof.
(ii) atypical antipsychotics including for example quetiapine and pharmaceutically active isomer(s) and metabolite(s) thereof.
(iii) antipsychotics including for example amisulpride, aripiprazole, asenapine, benzisoxidil, bifeprunox, carbamazepine, clozapine, chlorpromazine, debenzapine, divalproex, duloxetine, eszopiclone, haloperidol, iloperidone, lamotrigine, loxapine, mesoridazine, olanzapine, paliperidone, perlapine, perphenazine, phenothiazine, phenylbutylpiperidine, pimozide, prochlorperazine, risperidone, sertindole, sulpiride, suproclone, suriclone, thioridazine, trifluoperazine, trimetozine, valproate, valproic acid, zopiclone, zotepine, ziprasidone and equivalents and pharmaceutically active isomer(s) and metabolite(s) thereof.
(iv) anxiolytics including for example alnespirone, azapirones, benzodiazepines, barbiturates such as adinazolam, alprazolam, balezepam, bentazepam, bromazepam, brotizolam, buspirone, clonazepam, clorazepate, chlordiazepoxide, cyprazepam, diazepam, diphenhydramine, estazolam, fenobam, flunitrazepam, flurazepam, fosazepam, lorazepam, lormetazepam, meprobamate, midazolam, nitrazepam, oxazepam, prazepam, quazepam, reclazepam, tracazolate, trepipam, temazepam, triazolam, uldazepam, zolazepam and equivalents and pharmaceutically active isomer(s) and metabolite(s) thereof.
(v) anticonvulsants including for example carbamazepine, clonazepam, ethosuximide, felbamate, fosphenyloin, gabapentin, lacosamide, lamotrogine, levetiracetam, oxcarbazepine, phenobarbital, phenyloin, pregabaline, rufinamide, topiramate, valproate, vigabatrine, zonisamide and equivalents and pharmaceutically active isomer(s) and metabolite(s) thereof.
(vi) Alzheimer's therapies including for example donepezil, rivastigmine, galantamine, memantine, and equivalents and pharmaceutically active isomer(s) and metabolite(s) thereof.
(vii) Parkinson's therapies including for example deprenyl, L-dopa, Requip, Mirapex, MAOB inhibitors such as selegine and rasagiline, comP inhibitors such as Tasmar, A-2 inhibitors, dopamine reuptake inhibitors, NMDA antagonists, Nicotine agonists, Dopamine agonists and inhibitors of neuronal nitric oxide synthase and equivalents and pharmaceutically active isomer(s) and metabolite(s) thereof.
(viii) migraine therapies including for example almotriptan, amantadine, bromocriptine, butalbital, cabergoline, dichloralphenazone, dihydroergotamine, eletriptan, frovatriptan, lisuride, naratriptan, pergolide, pizotiphen, pramipexole, rizatriptan, ropinirole, sumatriptan, zolmitriptan, zomitriptan, and equivalents and pharmaceutically active isomer(s) and metabolite(s) thereof.
(ix) stroke therapies including for thrombolytic therapy with eg activase and desmoteplase, abciximab, citicoline, clopidogrel, eptifibatide, minocycline, and equivalents and pharmaceutically active isomer(s) and metabolite(s) thereof.
(x) urinary incontinence therapies including for example darafenacin, falvoxate, oxybutynin, propiverine, robalzotan, solifenacin, tolterodine and equivalents and pharmaceutically active isomer(s) and metabolite(s) thereof.
(xi) neuropathic pain therapies including for example lidocain, capsaicin, and anticonvulsants such as gabapentin, pregabalin, and antidepressants such as duloxetine, venlafaxine, amitriptyline, klomipramine, and equivalents and pharmaceutically active isomer(s) and metabolite(s) thereof.
(xii) nociceptive pain therapies such as paracetamol, NSAIDS and coxibs, such as celecoxib, etoricoxib, lumiracoxib, valdecoxib, parecoxib, diclofenac, loxoprofen, naproxen, ketoprofen, ibuprofen, nabumeton, meloxicam, piroxicam and opioids such as morphine, oxycodone, buprenorfin, tramadol, and equivalents and pharmaceutically active isomer(s) and metabolite(s) thereof.
(xiii) insomnia therapies including for example agomelatine, allobarbital, alonimid, amobarbital, benzoctamine, butabarbital, capuride, chloral, cloperidone, clorethate, dexclamol, ethchlorvynol, etomidate, glutethimide, halazepam, hydroxyzine, mecloqualone, melatonin, mephobarbital, methaqualone, midaflur, nisobamate, pentobarbital, phenobarbital, propofol, ramelteon, roletamide, triclofos, secobarbital, zaleplon, zolpidem and equivalents and pharmaceutically active isomer(s) and metabolite(s) thereof.
(xiv) mood stabilizers including for example carbamazepine, divalproex, gabapentin, lamotrigine, lithium, olanzapine, quetiapine, valproate, valproic acid, verapamil, and equivalents and pharmaceutically active isomer(s) and metabolite(s) thereof.

Such combination products employ (1S)-1-(2-(difluoromethyl)pyridin-4-yl)-4-fluoro-1-(3-(pyrimidin-5-yl)phenyl)-1H-isoindol-3-amine hemifumarate within the dosage range described herein and the other pharmaceutically active compound or compounds within approved dosage ranges and/or as determined by a person skilled in the art.

Compounds of the present invention may be administered orally, parenteral, buccal, vaginal, rectal, inhalation, insufflation, sublingually, intramuscularly, subcutaneously, topically, intranasally, intraperitoneally, intrathoracially, intravenously, epidurally, intrathecally, intracerebroventricularly and by injection into the joints.

The dosage will depend on the route of administration, the severity of the disease, age and weight of the patient and other factors normally considered by the attending physician, when determining the individual regimen and dosage level as the most appropriate for a particular patient.

An effective amount of a compound of the present invention for use in therapy of dementia is an amount sufficient to symptomatically relieve in a warm-blooded animal, particularly a human the symptoms of dementia, to slow the progression of dementia, or to reduce in patients with symptoms of dementia the risk of getting worse.

In addition to the compounds of the present invention, the pharmaceutical composition of this invention may also contain, or be co-administered (simultaneously or sequentially) with, one or more pharmacological agents of value in treating one or more disease conditions referred to herein.

The quantity of (1S)-1-(2-(difluoromethyl)pyridin-4-yl)-4-fluoro-1-(3-(pyrimidin-5-yl)phenyl)-1H-isoindol-3-amine hemifumarate to be administered will vary for the patient being treated and will vary from about 10 ng/kg of body weight to 100 mg/kg of body weight per day and preferably will be from 10 ng/kg to 10 mg/kg per day. For instance, dosages can be readily ascertained by those skilled in the art from this disclosure and the knowledge in the art. Thus, the skilled artisan can readily determine the amount of compound and optional additives, vehicles, and/or carrier in compositions and to be administered in methods of the invention.

### Methods of Preparation

The present invention also relates to processes for preparing (1S)-1-(2-(difluoromethyl)pyridin-4-yl)-4-fluoro-1-(3-(pyrimidin-5-yl)phenyl)-1H-isoindol-3-amine hemifumarate.

Throughout the following description of such processes it is to be understood that, where appropriate, suitable protecting groups will be added to, and subsequently removed from the various reactants and intermediates in a manner that will be readily understood by one skilled in the art of organic synthesis. Conventional procedures for using such protecting groups as well as examples of suitable protecting groups are for example described in Protective Groups in Organic Synthesis by T. W. Greene, P. G. M Wutz, 3^{rd} Edition, Wiley-Interscience, New York, 1999. It is to be understood that microwaves can alternatively be used for the heating of reaction mixtures.

R¹⁴ is defined as aryl or as:

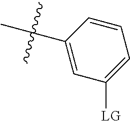

wherein LG represents a leaving group such as halogen (such as chlorine, bromine or iodine)

R^{C} is defined as:

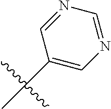

Said process comprises:

### (i) Formation of a Corresponding Compound of Formula (V):

A compound of formula (V) may be obtained as depicted in Scheme 1, for example by metallation or halogen metal exchange of a compound of formula (II), wherein G is either a hydrogen or a halogen respectively, to obtain an intermediate of formula (III), wherein M is a metal such as Zn or Mg, and L is a ligand such as halogen and n is between 0 and 6. The intermediate (III) may or may not be isolated and is subsequently reacted further with a compound of formula (IV), wherein LG is either N(CH₃)(OCH₃) or halogen or ethyl sulfide or another suitable leaving group for example as described by R. K. Dieter, (Tetrahedron, 55 (1999) 4177-4236).

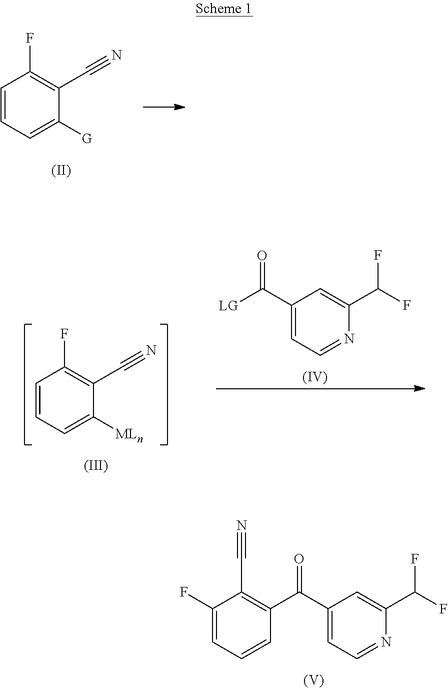

The reaction may be carried out by treating a compound of formula (II), wherein G is a halogen (such as iodine or bromine) with an appropriate metallating reagent, such as a lithium reagent (such as tert-butyllithium, n-butyllithium, lithium diispropylamide or lithium tetramethyl piperidine) or with a Grignard reagent (such as isopropylmagnesium bromide) or with a metal (such as magnesium, zinc or manganese), by standard methods known in the art. Optionally, the formed intermediate of formula (III) may be further transmetallated by treatment with a metal salt or metal complex, such as copper cyanide di(lithium bromide) or di(lithium chloride), to obtain a new intermediate of formula (III), and then treat said intermediate of formula (III) with a compound of formula (IV), wherein LG represents a leaving group such as a halogen (such as chlorine) or N(CH₃)(OCH₃) or ethyl sulfide. Optionally, this transformation may be performed under the influence of a transition metal catalyst, such as a palladium, copper, nickel or ruthenium salt or complex, for example as described in the literature by R. K. Dieter, (Tetrahedron, 55 (1999) 4177-4236). The reaction may be performed in a suitable solvent such as acetonitrile, toluene, diethyl ether or tetrahydrofuran at a temperature between −105° C. and 70° C.

### (ii) Formation of a Corresponding Compound of Formula (VIII):

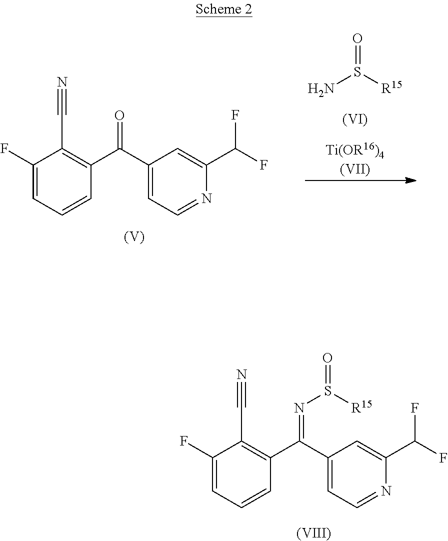

A compound of formula (VIII) may be obtained by reacting a compound of formula (V) with a compound of formula (VI) (Scheme 2), wherein R¹⁵ is alkyl (such as for example tert-butyl). Compound (VI) can be either a racemate or an enantiomerically enriched or enantiopure compound. The reaction is performed in the presence of a suitable Lewis acid of formula (VII), wherein R¹⁶ is alkyl (such as ethyl or isopropyl). The reaction is performed in a suitable solvent (such as diethyl ether, dichloromethane, tetrahydrofuran, or 2-methyltetrahydrofuran) at a temperature between room temperature and reflux temperature. The product compound VIII in solution is purified via an aqueous work-up, by extracting the titanium salts with aqueous acid, preferably sulfuric acid. If compound (VI) is an optically pure enantiomer, the enantiomerically pure compound (VIII) may be obtained.

(iii) Formation of a Corresponding Compound of Formula (X)

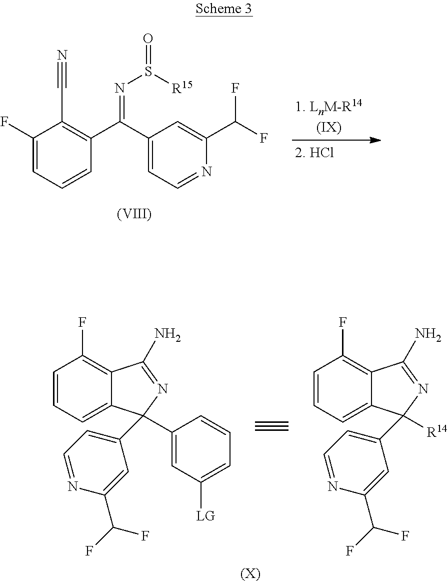

A compound of formula (X) may be prepared as shown in Scheme 3 by treating a compound of formula (VIII), with an appropriate organo metallic reagent of formula (IX) wherein M is a metal (such as lithium zinc or magnesium), L is a ligand (such as halogen or R¹⁴) and n is between 0 and 2, and R¹⁴ is as defined above, followed by treatment with a suitable acid, such as hydrochloric acid. The reaction may be performed in a suitable solvent, such as diethyl ether, toluene or tetrahydrofuran, at a temperature between −105° C. and room temperature. The organo metallic reagent of formula (IX) may be generated from the corresponding LG-R¹⁴, wherein LG represents a leaving group such as a halogen, such as iodide, bromide or chloride by known methods as described in Advanced Organic Chemistry by Jerry March 4th edition, Wiley Interscience. If enantiomerically pure or enriched compound (VIII) is used in this reaction, an enantiomerically pure or enantiomerically enriched compound (X) might be obtained.

### (iv) Formation of a Corresponding Compound of Formula (I):

A compound of formula (I) may be obtained (Scheme 4) by starting from, for example, a compound of formula (Xa), wherein LG represents a leaving group such as halogen (such as chlorine, bromine or iodine) or an alkyl-, aryl- or haloalkyl-sulfonate (such as triflate), and reacting said compound (Xa) with a compound of formula (XI), wherein R^{C} is defined as above and T represents a boronic acid or a boronic ester or a stannane or a zinc halide or zinc-R^{C}, under the influence of a transition metal catalyst as described in for example Metal Catalyzed Cross-coupling Reactions by F. Diederich and P. J. Stang, Wiley V C H, Weinheim, 1998. The compound of formula (XI) may be generated from the corresponding LG-R^{C}, wherein LG represents a leaving group, such as a halogen, (such as iodide, bromide or chlorine) by known methods as described in for example Advanced Organic Chemistry by Jerry March 4th edition, Wiley Interscience,

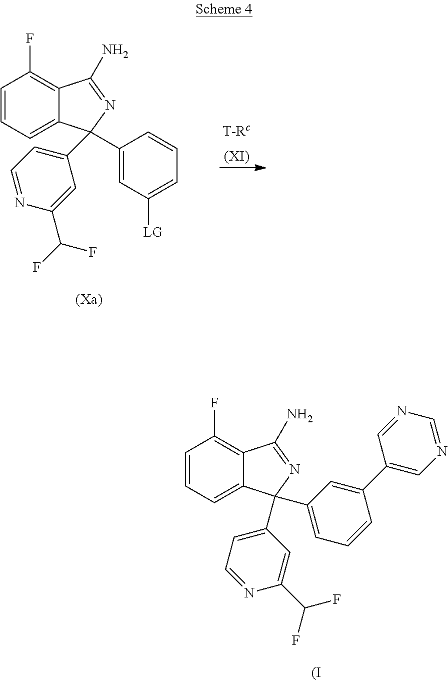

The reaction may be carried out by coupling of a compound of formula (Xa), with an appropriate aryl or heteroaryl boronic acid or boronic ester or stannane or a zinc halide or zinc-R^{C} of formula T-R^{C}. The reaction may also be carried out using a suitable metal catalyst such as a palladium catalyst, such as [1,1′-bis(diphenylphosphino)ferrocene]palladium(II) chloride, tetrakis(triphenylphosphine)-palladium(0), palladium diphenylphosphineferrocene dichloride, palladium(II) acetate or bis(dibenzylideneacetone) palladium (0). Optionally, a suitable ligand such as triphenylphosphine, tri-tert-butylphosphine, 3-(di-tert-butylphosphonium)propane sulfonate or 2-(dicyclohexylphosphino)biphenyl is employed. A suitable base, such as cesium fluoride, an alkyl amine, such as triethyl amine, or an alkali metal or alkaline earth metal carbonate or hydroxide such as potassium carbonate, sodium carbonate, cesium carbonate, or sodium hydroxide, may be used in the reaction. Said reaction may be performed at a temperature range between +20° C. and +160° C., in a suitable solvent, such as toluene, tetrahydrofuran, dioxane, dimethoxyethane, water, ethanol, 2-propanol, N,N-dimethylacetamide or N,N-dimethylformamide, or mixtures thereof. If enantiomerically pure or enriched compound (Xa) is used in this reaction, an enantiomerically pure or enantiomerically enriched compound (I) might be obtained.

(v) Formation of a Fumaric Acid Salt from (I) of the Formula XII:

A salt of formula (XII) may be obtained (Scheme 5) by starting from a solution of (I) in a suitable solvent, for example ethyl acetate, isopropyl acetate or n-butanol followed by mixing the obtained solution with fumaric acid (XIII) dissolved in a suitable solvent, for example methanol or water, at a temperature between room temperature and 100° C. Optionally, fumaric acid and (I) may be sequentially dissolved or slurried in a suitable solvent or solvent mixture, for example ethyl acetate, ethyl acetate/methanol, isopropylacetate or isopropylacetate/methanol at a temperature between room temperature and 50° C. Crystallization may be obtained by evaporation of solvent and/or by cooling the solution.

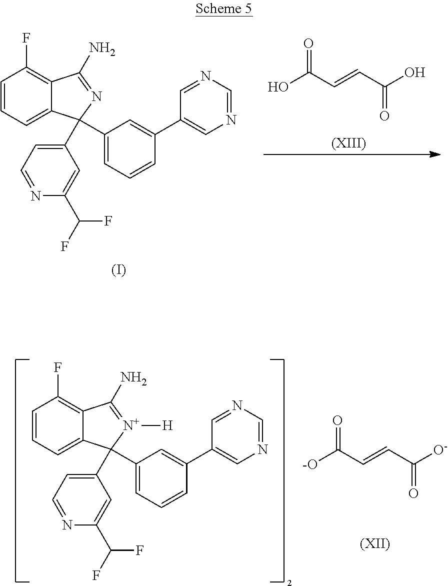

Compounds of formula (II), (III), (IV), (VI), (VII), (IX) and (XI) are commercially available compounds, or they are known in the literature, or they are prepared by standard processes known in the art.

More specifically, (1S)-1-(2-(difluoromethyl)pyridin-4-yl)-4-fluoro-1-(3-(pyrimidin-5-yl)phenyl)-1H-isoindol-3-amine can be prepared as shown in Scheme 6:

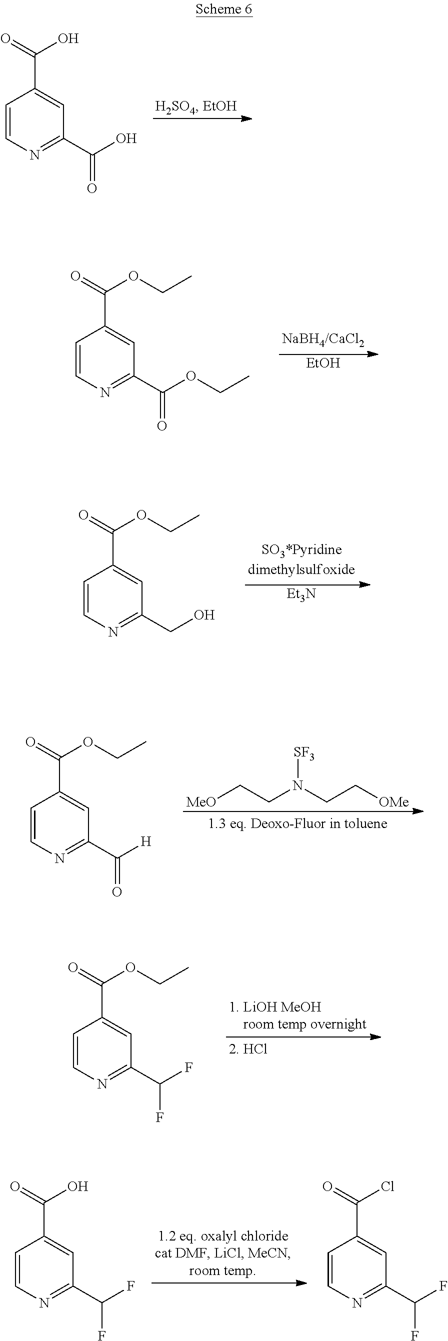

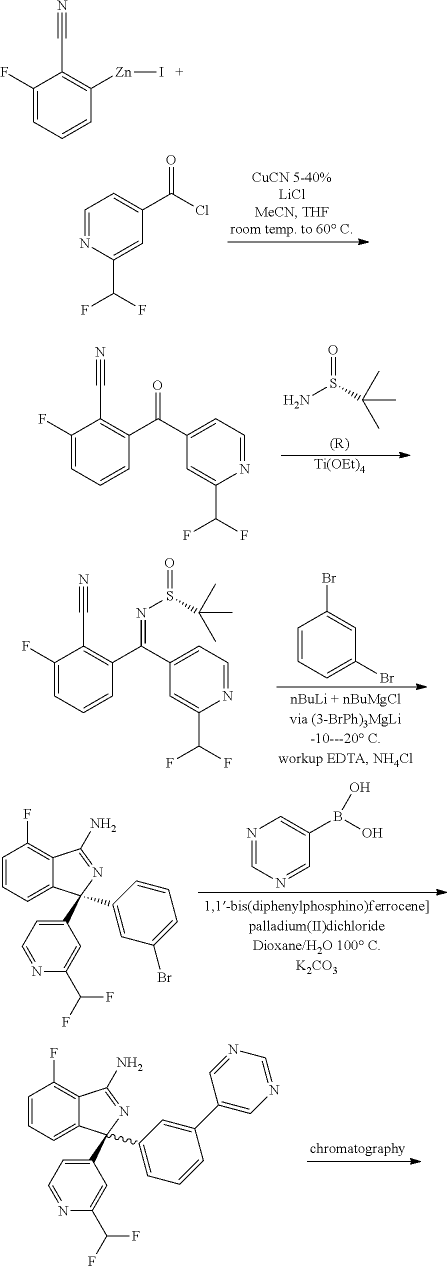

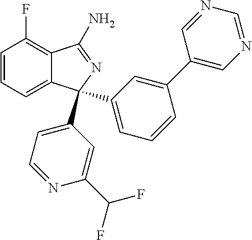

### Chromatographic separation of a mixture of (1S)-1-(2-(difluoromethyl)pyridin-4-yl)-4-fluoro-1-(3-(pyrimidin-5-yl)phenyl)-1H-isoindol-3-amine and (1R)-1-(2-(difluoromethyl)pyridin-4-yl)-4-fluoro-1-(3-(pyrimidin-5-yl)phenyl)-1H-isoindol-3-amine

The racemate or pseudoracemate of 1-(2-(difluoromethyl)pyridin-4-yl)-4-fluoro-1-(3-(pyrimidin-5-yl)phenyl)-1H-isoindol-3-amine can be dissolved in a Acetonitrile/Methanol mixture. The components are separated using either simulated moving bed liquid chromatography or batch liquid chromatography. The column stationary phase is Amylose tris-(5-chloro-2-methylphenylcarbamate) coated on silica gel, tradenames are Chiralpak AY (Daicel) and Amylose Lux-2 (Phenomenex). Acetonitrile/Methanol mixture is used as mobile phase.

### XRPD Instrumentation

An X-Ray Powder Diffraction (XRPD) pattern was collected under ambient conditions on a PANalytical X'Pert PRO MPD theta-theta system using long-fine-focus Cu Kα-radiation, wavelength of X-rays 1.5418 Å, at 45 kV and 40 mA. A programmable divergence slit and a programmable anti-scatter slit giving an irradiated length of 10 mm were used. 0.02 radian Soller slits were used on the incident and on the diffracted beam path. A 20 mm fixed mask was used on the incident beam path and a Nickel-filter was placed in front of a PIXcel-detector using 255 active channels. A thin flat sample was prepared on a flat zero background plate made of silicon using a spatula. The plate was mounted in a sample holder and rotated in a horizontal position during measurement. A diffraction pattern was collected between 2° 2theta and 40° 2theta in a continuous scan mode. Total time for the scan was approximately 10 minutes. A person skilled in the art can transform Cu Kα-radiation 2theta-values to d-values and vice versa.

### Peak Search on X-Ray Powder Diffraction Data

A peak search was made within the X'Pert High Score Plus software version 2.0, preceded by a background determination and withdrawal as well as angle correction against the internal standard of α-Al₂O₃. The peak positions were then manually adjusted. No K_{α2}-stripping was done.

### EXAMPLES

### Example 1

### Preparation of 3-fluoro-2-cyanophenyl (2-(difluoromethyl)pyridin-4-yl) ketone

In vessel 1,2-difluoromethyl-pyridinyl-4-yl carboxylic acid (20.0 g, 111 mmol) was slurried in toluene (200 mL) at 40° C. Oxalyl chloride (10.6 mL, 122 mmol) was added during 3 h. Dimethylformamide (70 mg) was used as a catalyst. After approximately 17 hours the formed 2-difluoromethyl-pyridinyl-4-yl carboxylic acid chloride solution was reduced by distillation to remove excess oxalyl chloride and hydrochloric acid. To vessel 2 was added 2-bromo-6-fluorobenzonitrile (26.9 g, 133 mmol) dissolved in tetrahydrofuran (THF, 60 ml) followed by cooling to −15° C. Isopropylmagnesium chloride (2.00 M in THF, 75 ml, 136 mmol) was added during approximately 30 minutes. The 2-difluoromethyl-pyridinyl-4-yl carboxylic acid chloride solution in vessel 1 was diluted with THF (20 mL) and added to vessel 2 over 5 minutes. 40 mL THF was used for rinsing. The reaction was run for approximately 16 h at −15° C., then 0° C. for 2 h. The mixture was cooled to −15° C. and quenched with acetic acid (13 mL, 227 mmol) and diluted with water (60 mL). The phases were separated at 45° C. and the organic layer concentrated until approximately 150 mL was remaining Isopropyl alcohol (IPA, 100 mL) was added and the mixture concentrated to approximately 150 mL. IPA (100 mL) was added followed by concentration to approximately 100 mL. IPA (60 ml) was added and the title product crystallized by cooling to 0° C. The product was filtered and washed with cool IPA (120 mL), then dried to give 23.6 g (76% yield) at 99% assay.

¹H NMR (400 MHz, DMSO-d₆) δ ppm 8.97 (d, 1h), 7.97-7.86 (m, 4H), 7.65 (d, 1H), 7.11 (t, 1H).

### Example 2

### Preparation of (R)-3-fluoro-2-cyanophenyl (2-(difluoromethyl)pyridin-4-yl) N-tert-butylsulfinyl imine

### Reaction

In vessel 1, 3-fluoro-2-cyanophenyl (2-(difluoromethyl)pyridin-4-yl) ketone (20.09 g, 73.4 mmol), (R)-(+)-2-methyl-2-propanesulfinamide (10.71 g, 86.6 mmol) and Ti(OEt)₄ (42.95 g, 188.3 mmol) were dissolved in 2-methyltetrahydrofuran (100 mL) and heated to reflux. After 3 hours the reaction mixture was cooled to 20° C.

To vessel 2 was added sulfuric acid (16.4 g, 167.1 mmol) and sodium sulfate (25.3 g, 176.2 mmol) and it was dissolved in water (143 mL). The mixture was then cooled to 12° C. The reaction solution from vessel 1 was added slowly to vessel 2 under vigorous stirring. 2-Methyltetrahydrofuran (20.0 mL) was used for rinsing. The temperature was adjusted to 20° C. and the mixture was left under mixing until all precipitations were dissolved (0.5 h). The water phase was separated off.

To vessel 3 was added sulfuric acid (4.1 g, 41.8 mmol) and sodium sulfate (6.3 g, 44.0 mmol) and it was dissolved in water (36 mL). The sulfuric acid solution from vessel 3 was added under mixing to the organic phase in vessel 2. The temperature was adjusted to 20° C. and the mixture was left under mixing until all precipitations were dissolved (0.3 h). The water phase was separated off.

The organic phase was washed with ammonium acetate (14.0 g, 181.6 mmol) dissolved in water (32 mL) at 10° C. and then washed with sodium chloride (10.1 g, 172.8 mmol) dissolved in water (40 mL) at 20° C. The organic phase was concentrated until 60 mL remaining Toluene (120 mL) was added. The solution was concentrated until 46 mL remaining Toluene (134 mL) was added. The solution was clear filtered.

Assay 13.9 wt %

Yield 82.9%

¹H NMR (400 MHz, DMSO-d₆) δ ppm 8.87 (d, 1h), 7.90 (br s, 1H), 7.78 (s, 1H), 7.71 (t, 1H), 7.63 (d, 1H), 7.55 (br s, 1H), 7.09 (t, 1H), 1.32 (s, 9H)

### Example 3

### Preparation of 1-(3-bromophenyl)-1-(2-(difluoromethyl)pyridin-4-yl)-4-fluoro-1H-isoindol-3-amine hemifumarate

n-Butyl lithium (2.5M, 21.7 mL, 54.2 mmol) and tetrahydrofuran (THF, 28 ml) were cooled to approximately −5° C. (inner temp., Ti) followed by addition of butylmagnesium chloride (20% w/w, 12.7 mL, 25.5 mmol) over approximately 14 minutes, then stirred for approximately 70 minutes at Ti=−3 to 0° C. 1,3-Dibromobenzene (19.5 g, 10.0 mL, 80.0 mmol) was added over approximately 20 minutes, Ti being max −2° C. After another hour (Ti approx. −2° C.) the mantle temperature (Tm) was set to −30° C. At Ti=−15° C. was added a toluene solution of (R)-3-fluoro-2-cyanophenyl (2-(difluoromethyl)pyridin-4-yl) N-tert-butylsulfinyl imine (49.9% w/w, 27.6 g, 13.8 g at 100%, 36.3 mmol) over approximately 50 minutes, Ti=−15° C. Toluene (13.8 mL) was used for rinsing. The reaction was stirred for approximately 1 hour 10 minutes, Ti was −23° C. at the end. Ethylenediaminetetraacetic acid (EDTA) (0.54 g, 1.85 mmol) was added and then aqueous ammonium chloride (9.6 g NH₄Cl in 55 mL water) over approximately 25 minutes. The mantle temperature was set to 25° C. Isopropyl acetate (iPrOAc, 117 mL) was added followed by extraction and separation of the aqueous phase. The organic layer was washed with aqueous sodium chloride (8.2 g NaCl in 33 mL water). The layers were separated. The combined aqueous layers were further extracted with iPrOAc (70 mL). This organic phase was washed with aq. NaCl (8.2 g NaCl in 33 mL water). The combined organic phases were charged with EDTA (0.65 g, 2.22 mmol) and left over night (break point). Diluted hydrogen chloride (36% w/w, 14 mL in water 41 mL) was added and the mixture cooled in a 10° C. bath. The layers were separated and the aq. phase saved. To the organic phase was added methanol (14 ml) and diluted hydrogen chloride (HCl, 36% w/w, 14 mL in water 27 ml). The mantle was set to 20° C. and the layers were stirred for approximately 1.5 hours. The aqueous phase was separated and the aqueous layer collected. The organic layer was extracted with aqueous HCl (36% w/w, 7 mL in water 41 mL). All three acidic aqueous layers were combined (T-mantle set to 0° C.). iPrOAc (approximately 55 ml) was added. Sodium hydroxide (50% w/w, 22 ml) was added. The layers were separated (Ti=13° C.), the organic layer collected and the aqueous layer further extracted with iPrOAc (55 mL). The combined organic layers were washed with sodium chloride (8.2 g) in water (33 mL). The organic layer was separated, dried over Na₂SO₄, filtered and distilled to 65 mL. iPrOAc was added to obtain a final solution of title compound (110 ml, 99.6 g, 12.6% w/w by NMR, yield 80%).

### Hemifumarate Preparation:

Fumaric acid (1.71 g, 14.6 mmol) was dissolved in methanol (40 mL) at ambient temperature. The solution of 1-(3-bromophenyl)-1-(2-(difluoromethyl)pyridin-4-yl)-4-fluoro-1H-isoindol-3-amine in iPrOAc from above (12.6% w/w, 12.5 g at 100%, 28.9 mmol) was added over 16 minutes followed by rinsing with iPrOAc (25 ml). Solid formation had started. The methanol was distilled off. iPrOAc (50 mL) was added followed by further distillation. The remaining slurry was charged with iPrOAc (75 mL) and then cooled from 20° C. to 0° C. over 1 hour and then stirred at 0° C. for 1 hour. The solids were filtered, washed with cooled iPrOAc (30 mL) and dried under vacuum at 50° C. for 3 hours. Yield 13.66 g, NMR-assay 97.1% w/w, 94% corrected yield.

### Example 4

### Preparation of 1-(3-bromophenyl)-1-(2-(difluoromethyl)pyridin-4-yl)-4-fluoro-1H-isoindol-3-amine

n-Butyl lithium (2.5M, 38.0 ml, 95.0 mmol) was cooled to approximately −10 to −20° C. followed by addition of butylmagnesium chloride (20% w/w, 25.9 ml, 44.3 mmol) then stirred for approximately 20 minutes. The mantle temperature was set to −33° C. followed by addition of 1,3-dibromobenzene (33.9 g, 17.4 mL, 139.3 mmol) over approximately 20 minutes, Ti being max −8.5° C. After another 20 minutes (Ti=−32° C.) was added a toluene solution of (R)-3-fluoro-2-cyanophenyl (2-(difluoromethyl)pyridin-4-yl) N-tert-butylsulfinyl imine (26.7% w/w, 90.0 g, 63.3 mmol) over approximately 20 minutes, Ti max −18° C. The inner temperature was set to −26° C. and the reaction stirred for 2 hours. Ethylenediaminetetraacetic acid (EDTA) (0.93 g, 3.2 mmol) was added followed by stirring for approximately 20 minutes. The mantle temperature was set to 25° C. Isopropyl acetate (iPrOAc, 216 mL) was added followed by EDTA (0.93 g, 3.2 mmol) and aqueous ammonium chloride (15% w/w, 96 mL). Another portion of iPrOAc (216 mL) was added followed by separation of the layers. The obtained organic layer 1 was washed with brine. The combined aqueous layers were extracted with iPrOAc (120 mL), separated and the organic layer washed with brine to obtain organic layer 2. Organic layers 1 and 2 were combined and EDTA (1.57 g, 5.4 mmol) was added followed by iPrOAc (96 mL) and then careful addition of diluted hydrogen chloride (36% w/w, 24.0 mL in water 72 mL). The layers were separated and the aqueous layer collected and treated with methanol (24 mL, 594 mmol) then diluted hydrogen chloride (HCl, 36% w/w, 24.0 mL in water 48 mL). The mantle was set to 20° C. and the layers were separated and the aqueous layer collected. The organic layer was extracted with aqueous HCl (36% w/w, 10.0 mL in water 40 mL). The two acidic aqueous layers were combined (T-mantle set to −10° C.), iPrOAc (96 mL) was added then sodium hydroxide (50% w/w, 40.0 mL, 734 mmol). The layers were separated, the organic layer collected and the aqueous layer further extracted with iPrOAc (96 mL). The two combined organic layers were washed with sodium chloride (19 g) in water (58 mL). The organic layer was separated and distilled to obtain a solution of the title compound (101 g, 20% w/w, yield 74%, enantiomeric excess 70% i.e. 85:15 desired:non-desired enantiomer).

### Example 5

### (1S)-1-(2-(difluoromethyl)pyridin-4-yl)-4-fluoro-1-(3-(pyrimidin-5-yl)phenyl)-1H-isoindol-3-amine

Inert atmosphere (nitrogen) was used throughout the reaction. 1-(3-bromophenyl)-1-(2-(difluoromethyl)pyridin-4-yl)-4-fluoro-1H-isoindol-3-amine hemifumarate (1.51 g, 2.66 mmol) and 5-pyrimidineboronic acid (0.43 g, 2.92 mmol) were dissolved in n-butanol (10.5 mL). Aqueous potassium carbonate (K₂CO₃, 1.64 g, 11.9 mmol in 4.5 mL water) was added, then 3-(di-tert-butylphosphonium)propane sulfonate (DTBPPS, 7.4 mg, 0.027 mmol) and palladium(II) diacetate (6.2 mg, 0.028 mmol). The formed two phase system was heated to 80° C. (bath) over 30 minutes, and then stirred at this temperature over night (it had completed after approximately 3 h). The reaction mixture was cooled to 50° C. over 30 min. n-Butanol (4 mL) was added. The lower aqueous phase was separated off and the organic layer washed with brine (10% w/w, 7 mL). The organic phase was filtered through a PALL syringe filter (0.8 μm) which was rinsed with n-butanol (4 mL). The collected organic phase was distilled to a final weight of 13.75 g with solid material present. This was heated in 90° C. and stirred for approximately 1 hour then the heating was switched off and the mixture slowly cooled in the bath. The mixture was then cooled on an ice-bath for approximately 1 hour. The solids were filtered off, washed with cool 1-butanol (2*5 mL) and dried to give 0.32 g 28% of a racemic mixture of the title compound. The mother liquors, 17.65 g n-butanol solution contained 3.8% w/w equivalent to 0.671 g (59% yield) of the title compound with an enantiomeric excess of 93.6% (3.2% opposite enantiomer).

### Example 6

### Preparation of (1S)-1-(2-(difluoromethyl)pyridin-4-yl)-4-fluoro-1-(3-(pyrimidin-5-yl)phenyl)-1H-isoindol-3-amine hemifumarate Form A

0.04348 g (1S)-1-(2-(difluoromethyl)pyridin-4-yl)-4-fluoro-1-(3-(pyrimidin-5-yl)phenyl)-1H-isoindol-3-amine was dissolved in 10.0 mL 0.00502 M fumaric acid in ethyl acetate at 50° C.

Part of the ethyl acetate was evaporated under a nitrogen gas stream at 50° C.

After 16 days of shaking at 50° C. in an Eppendorf Thermomixer, 750 rpm, a white precipitation was seen on the walls of the 15 mL test tube above the surface. Manual shaking quickly dissolved the main part of it. A new evaporation under nitrogen gas flow followed by further shaking at 50° C. for 6 days prior to centrifuge filtration of part of the slurry gave a solid first sample. Next day most of the remaining solid, the second sample, was separated by centrifuge filtration.

### Example 7

### Preparation of (1S)-1-(2-(difluoromethyl)pyridin-4-yl)-4-fluoro-1-(3-(pyrimidin-5-yl)phenyl)-1H-isoindol-3-amine hemifumarate Form A

0.45 g of (1S)-1-(2-(difluoromethyl)pyridin-4-yl)-4-fluoro-1-(3-(pyrimidin-5-yl)phenyl)-1H-isoindol-3-amine was dissolved in 3 mL of ethyl acetate. 60 mg of fumaric acid was dissolved in approximately 5 mL of methanol at room temperature. The clear solutions were mixed and subsequently evaporated to dryness. 5 mL of ethyl acetate was added to the amorphous material obtained and left on mixing for one week. The thick slurry obtained was then filtered and the solid was left on drying for 48 hours at 40° C. and vacuum.

### Example 8

### Preparation of (1S)-1-(2-(difluoromethyl)pyridin-4-yl)-4-fluoro-1-(3-(pyrimidin-5-yl)phenyl)-1H-isoindol-3-amine hemifumarate Form A

Fumaric acid (78 mg, 0.665 mmol) was dissolved in methanol (4 mL) at ambient temperature. (1S)-1-(2-(difluoromethyl)pyridin-4-yl)-4-fluoro-1-(3-(pyrimidin-5-yl)phenyl)-1H-isoindol-3-amine dissolved in n-butanol (15.08 g solution, assay 3.80% w/w, 1.33 mmol) was added dropwise over a few minutes. n-Butanol (2*2 mL) was used for complete transfer. The methanol was removed and volume reduced by distillation to 8.3 g of n-butanol solution which was heated to 90° C. (mantle=Tₘ). The amount was adjusted to 9.5 g by adding n-butanol (1.5 mL) followed by heating to Tₘ=100° C. When the inner temperature (Tᵢ) was 87° C. the mixture was cooled from Tₘ=100 to 10° C. over 3 hours the left stirring at 10° C. over night. The solids were filtered and washed with ice-cooled n-butanol (2*4 mL) then dried at 50° C. under vacuum. Yield 500 mg, 72%, NMR assay 82.6% w/w (free base) 93.7% w/w (hemifumarate), HPLC: purity 98.3%, enantiomeric purity 99.5% (0.5% opposite enantiomer), XRPD in agreement with reference for Form A.

### Assays

The level of activity of (1S)-1-(2-(Difluoromethyl)pyridin-4-yl)-4-fluoro-1-(3-(pyrimidin-5-yl)phenyl)-1H-isoindol-3-amine hemifumarate can be tested using the following methods:

### TR-FRET Assay

The β-secretase enzyme used in the TR-FRET is prepared as follows:

The cDNA for the soluble part of the human β-Secretase (AA 1-AA 460) is cloned using the ASP2-Fc10-1-IRES-GFP-neoK mammalian expression vector. The gene is fused to the Fc domain of IgG1 (affinity tag) and stably cloned into HEK 293 cells. Purified sBACE-Fc is stored in −80° C. in Tris buffer, pH 9.2 and has a purity of 95%. The enzyme (truncated form) is diluted to 6 μg/mL (stock 1.3 mg/mL) and the substrate (Europium)CEVNLDAEFK(Qsy7) to 200 nM (stock 120 μM) in reaction buffer (NaAcetate, chaps, triton x-100, EDTA pH4.5). The robotic systems Biomek FX and Velocity 11 are used for all liquid handling and the enzyme and substrate solutions are kept on ice until they are placed in the robotic system. Enzyme (9 μl) is added to the plate then 1 μl of compound in dimethylsulphoxide is added, mixed and pre-incubated for 10 minutes. Substrate (10 μl) is then added, mixed and the reaction proceeded for 15 minutes at room temperature. The reaction is stopped with the addition of Stop solution (7 μl, NaAcetate, pH 9). The fluorescence of the product is measured on a Victor II plate reader with an excitation wavelength of 340 nm and an emission wavelength of 615 nm. The assay is performed in a Costar 384 well round bottom, low volume, non-binding surface plate (Corning #3676). The final concentration of the enzyme is 2.7 μg/ml; the final concentration of substrate is 100 nM (Km of ˜250 nM). The dimethylsulphoxide control, instead of test compound, defines the 100% activity level and 0% activity is defined by wells lacking enzyme (replaced with reaction buffer). A control inhibitor is also used in dose response assays and has an IC50 of ˜575 nM.

The (1S)-1-(2-(Difluoromethyl)pyridin-4-yl)-4-fluoro-1-(3-(pyrimidin-5-yl)phenyl)-1H-isoindol-3-amine hemifumarate Form A had an average IC50 of 63.2 nM in this assay.

sAPPβ Release Assay

SH-SY5Y cells are cultured in DMEM/F-12 with Glutamax, 10% FCS and 1% non-essential aminoacids and cryopreserved and stored at −140° C. at a concentration of 7.5×10⁶ cells per vial. Thaw cells and seed at a conc. of 1.5×10⁵/ml in DMEM/F-12 with Glutamax, 10% FCS and 1% non-essential aminoacids to a 96-well tissue culture treated plate, 100 μl cell susp/well. The cell plates are then incubated for 7 hours at 37° C., 5% CO2. The cell medium is removed, followed by addition of 90 μl compound diluted in DMEM/F-12 with Glutamax, 10% FCS, 1% non-essential aminoacids and 1% PeSt to a final conc. of 1% DMSO. The compound is incubated with the cells for 16 h (over night) at 37° C., 5% CO₂. Meso Scale Discovery (MSD) plates are used for the detection of sAPPβ release. MSD sAPPβ plates are blocked in 3% BSA in Tris wash buffer (150 μl/well) for 1 hour in RT and washed 4 times in Tris wash buffer (150 μl/well). 50 μl of medium is transferred to the pre-blocked and washed MSD sAPPβ microplates, and the cell plates are further used in an ATP assay to measure cytotoxicity. The MSD plates are incubated with shaking in RT for 1 hour followed by washing 4 times. 25 μl detection antibody is added (1 nM) per well followed by incubation with shaking in RT for 1 h and washing 4 times. 150 μl Read Buffer is added per well and the plates are read in a SECTOR Imager.

### ATP Assay

As indicated in the sAPPβ release assay, after transferring 50 μL medium from the cell plates for sAPPβ detection, the plates are used to analyse cytotoxicity using the ViaLight™ Plus cell proliferation/cytotoxicity kit from Cambrex BioScience that measures total cellular ATP. The assay is performed according to the manufacture's protocol. Briefly, 25 μL cell lysis reagent is added per well. The plates are incubated at room temperature for 10 min. Two min after addition of 50 μL reconstituted ViaLight™ Plus ATP reagent, the luminescence is measured in a Wallac Victor2 1420 multilabel counter.

1-23. (canceled)

24. (1S)-1-(2-(Difluoromethyl)pyridin-4-yl)-4-fluoro-1-(3-(pyrimidin-5-yl)phenyl)-1H-isoindol-3-amine hemifumarate.

25. (1S)-1-(2-(Difluoromethyl)pyridin-4-yl)-4-fluoro-1-(3-(pyrimidin-5-yl)phenyl)-1H-isoindol-3-amine hemifumarate Form A.

26. (1S)-1-(2-(Difluoromethyl)pyridin-4-yl)-4-fluoro-1-(3-(pyrimidin-5-yl)phenyl)-1H-isoindol-3-amine hemifumarate Form A characterized in providing an X-ray powder diffraction (XRPD) pattern, exhibiting substantially the following main peaks with d-values:
d-spacing [Å]
4.37

27. (1S)-1-(2-(Difluoromethyl)pyridin-4-yl)-4-fluoro-1-(3-(pyrimidin-5-yl)phenyl)-1H-isoindol-3-amine hemifumarate Form A characterized in providing an X-ray powder diffraction pattern, exhibiting substantially the following main peaks with d-values:
d-spacing [Å]
8.2
5.3
4.92
4.54
4.37

28. (1S)-1-(2-(Difluoromethyl)pyridin-4-yl)-4-fluoro-1-(3-(pyrimidin-5-yl)phenyl)-1H-isoindol-3-amine hemifumarate Form A characterized in providing an X-ray powder diffraction pattern, exhibiting substantially the following main peaks with d-values:
d-spacing [Å]
8.7
8.2
7.3
6.7
6.4
6.2
5.9
5.3
4.92
4.54
4.37
4.14

29. (1S)-1-(2-(Difluoromethyl)pyridin-4-yl)-4-fluoro-1-(3-(pyrimidin-5-yl)phenyl)-1H-isoindol-3-amine hemifumarate Form A characterized in providing an X-ray powder diffraction pattern, exhibiting substantially the following main peaks with d-values:
d-spacing [Å]
13.3
8.7
8.2
7.3
6.7
6.4
6.2
5.9
5.6
5.3
4.92
4.54
4.37
4.14

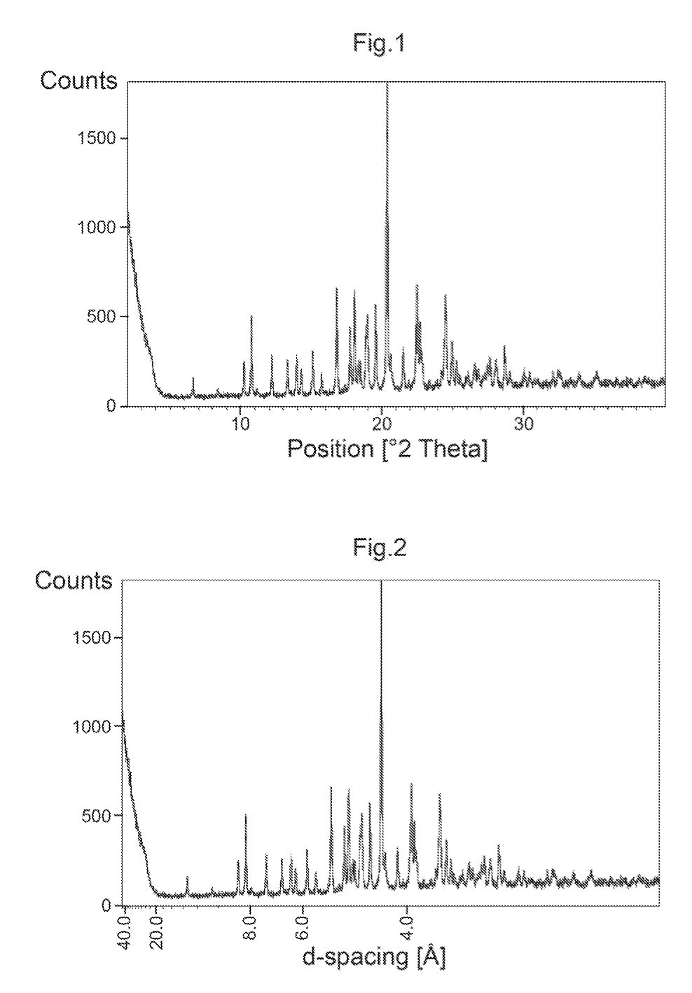

30. (1S)-1-(2-(Difluoromethyl)pyridin-4-yl)-4-fluoro-1-(3-(pyrimidin-5-yl)phenyl)-1H-isoindol-3-amine hemifumarate Form A characterized in providing an X-ray powder diffraction pattern essentially as shown in FIG. 1 or FIG. 2.

31. A pharmaceutical composition comprising as active ingredient a therapeutically effective amount of a salt according to any claim 24, in association with pharmaceutically acceptable excipients, carriers or diluents.

32. A method of treating or preventing an Aβ-related pathology in a patient in need thereof, comprising administering to said patient a therapeutically effective amount of a salt of claim 24.

33. The method of claim 32, wherein said Aβ-related pathology is Downs syndrome, a β-amyloid angiopathy, cerebral amyloid angiopathy, hereditary cerebral hemorrhage, a disorder associated with cognitive impairment, MCI (“mild cognitive impairment”), Alzheimer Disease, memory loss, attention deficit symptoms associated with Alzheimer disease, neurodegeneration associated with Alzheimer disease, dementia of mixed vascular origin, dementia of degenerative origin, pre-senile dementia, senile dementia, dementia associated with Parkinson's disease, progressive supranuclear palsy or cortical basal degeneration.

34. A method of treating or preventing Alzheimer's Disease in a patient in need thereof, comprising administering to said patient a therapeutically effective amount of a salt of claim 24.

35. A method of treating or preventing an Aβ-related pathology in a patient in need thereof, comprising administering to said patient a therapeutically effective amount of a salt of claim 24, and at least one cognitive enhancing agent, memory enhancing agent, or choline esterase inhibitor.

36. A process for the preparation of a compound of formula 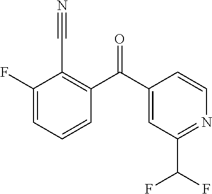
comprising reacting a compound of formula 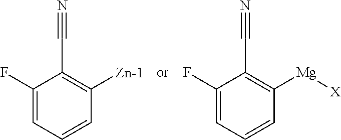
with a compound of formula 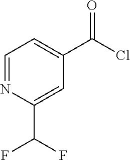
optionally in the presence of 0.1-40 mol % of CuCN or 0.1-40 mol % CuI or 0.1-40 mol % CuBr*SMe₂ or 0.1-40 mol % Li₂CuCl₄ or 0.1-40 mol % Pd(OAc)₂ or 0.1-40 mol % Dichloro(1,10-phenanthroline)palladium(II) or 0.1-40 mol % Dichloro(p-cymene)ruthenium(II)-Dimer or 0.1-40 mol % Ni(acetylacetonate)₂.

37. A process for the preparation of a compound of formula 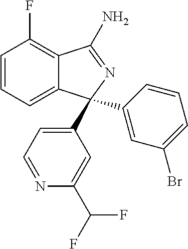
comprising treating a compound of formula 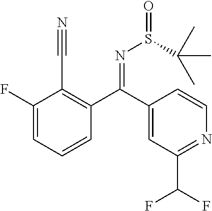
with an organo metallic reagent of formula LₙM-R¹⁴ wherein M is a metal, L is a ligand and n is 0, 1 or 2, and R¹⁴ is 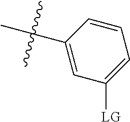
wherein LG represents a leaving group;
followed by treatment with acid.

38. A process according to claim 37, wherein
M is lithium, zinc or magnesium;
L is halogen or R¹⁴; and
LG is halogen.

39. A process for preparing a compound of formula (VIII), comprising
reacting a compound of formula (V) 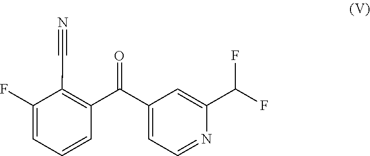
with a compound of formula (VI) in the presence of a compound of a compound of formula (VII) 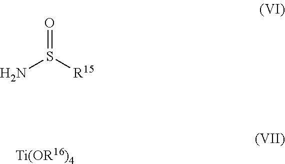
wherein R¹⁵ and R¹⁶ is independently alkyl;
and wherein the compound of formula (VIII) 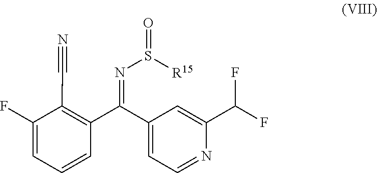
is obtained via isolation in solution following an aqueous work-up with aqueous acid.

40. A process according to claim 39, wherein the work-up is carried out using aqueous sulfuric acid.

41. A compound selected from
3-fluoro-2-cyanophenyl (2-(difluoromethyl)pyridin-4-yl) ketone;
3-fluoro-2-cyanophenyl (2-(difluoromethyl)pyridin-4-yl) N-tert-butylsulfinyl imine; and
(R)-3-fluoro-2-cyanophenyl (2-(difluoromethyl)pyridin-4-yl) N-tert-butylsulfinyl imine.
